(19) United States
(12) Patent Application Publication
(10) Pub. No.: US 2012/0253438 A1
(43) Pub. Date: Oct. 4, 2012

(54) COUPLING MECHANISMS FOR USE WITH A MEDICAL ELECTRICAL LEAD

(76) Inventors: Wei Gan, Woodbury, MN (US); Kevin R. Seifert, Forest Lake, MN (US); Zhongping Yang, Woodbury, MN (US)

(21) Appl. No.: 13/077,199

(22) Filed: Mar. 31, 2011

(51) Int. Cl. A61N 1/05

(52) U.S. Cl. 607/116

(57) ABSTRACT

An implantable medical lead may include components or mechanisms that can reduce the amount of induced current that is conducted to electrodes of the lead. A medical lead may, for example, have an energy dissipating structure that is connected to an electrode of the lead. This disclosure provides for coupling mechanisms to couple current induced on the lead to the energy dissipating structure. The coupling mechanisms described herein provide continuous contact with both electrode shaft and the energy dissipating structure while producing forces on the electrode shaft that is small enough to permit extension and retraction of the electrode from the lead.

### TECHNICAL FIELD

The present disclosure relates to coupling mechanisms for use with implantable medical electrical leads. More particularly, this disclosure describes coupling mechanism for connecting an energy dissipating structure of the electrical medical lead to an electrode of the medical electrical lead to redirect current induced on the lead by high frequency signals.

### BACKGROUND

In the medical field, implantable medical electrical leads are used with a wide variety of medical devices. For example, implantable medical electrical leads are commonly used to form part of an implantable medical system that provides therapeutic electrical stimulation to a patient, such as cardiac electrical stimulation to the heart in the form of pacing, cardioversion, defibrillation, or resynchronization pulses. The pulses can be delivered to the heart or other desired location within the patient via electrodes disposed on the leads, e.g., typically near distal ends of the leads. In that case, the leads may position the electrodes with respect to various locations so that the implantable medical system can deliver pulses to the appropriate locations. Leads are also used for sensing purposes, or for both sensing and stimulation purposes. Implantable leads are also used in neurological devices to deliver electrical stimulation to reduce the effects of a number of neurological disorders and in a number of other contexts.

Patients that have implantable medical systems may benefit, or even require, various medical imaging procedures to obtain images of internal structures of the patient. One common medical imaging procedure is magnetic resonance imaging (MRI). MRI procedures may generate higher resolution and/or better contrast images (particularly of soft tissues) than other medical imaging techniques. MRI procedures also generate these images without delivering ionizing radiation to the body of the patient, and, as a result, MRI procedures may be repeated without exposing the patient to such radiation.

During an MRI procedure, the patient or a particular part of the patient's body is positioned within an MRI device. The MRI device generates a variety of magnetic and electromagnetic fields to obtain the images of the patient, including a static magnetic field, gradient magnetic fields, and radio frequency (RF) fields. The static magnetic field may be generated by a primary magnet within the MRI device and may be present prior to initiation of the MRI procedure. The gradient magnetic fields may be generated by electromagnets of the MRI device and may be present during the MRI procedure. The RF fields may be generated by transmitting/receiving coils of the MRI device and may be present during the MRI procedure. If the patient undergoing the MRI procedure has an implantable medical system, the various fields produced by the MRI device may have an effect on the operation of the medical leads and/or the implantable medical device (IMD) to which the leads are coupled. For example, the gradient magnetic fields or the RF fields generated during the MRI procedure may induce energy on the implantable leads (e.g., in the form of a current), which may be conducted to tissue via the electrodes of the lead.

### SUMMARY

An implantable medical lead may include components or mechanisms that can reduce the amount of induced current that is conducted to electrodes of the lead. A medical lead may, for example, have an energy dissipating structure that is coupled to an electrode of the lead. This disclosure provides for coupling mechanisms to couple the energy dissipating structure to the electrode. The coupling mechanisms described herein provide continuous contact with both electrode shaft and the energy dissipating structure while producing force on the electrode shaft that is small enough to permit extension and retraction of the electrode from the lead.

In one example, the disclosure is directed to a medical electrical lead comprising a lead body having a proximal end configured to couple to an implantable medical device and a distal end. The lead also includes a conductive electrode shaft located near the distal end of the lead body, a conductor that extends from the proximal end of the lead body and couples to the conductive electrode shaft, and an electrode located near the distal end of the lead body and electrically coupled to an opposite end of the conductive electrode shaft as the conductor. The lead includes an energy dissipating structure located adjacent the electrode, the energy dissipating structure including a conductive element and a coupling mechanism formed of a conductive material that contacts the conductive electrode shaft and the energy dissipating structure. The conductive material of the coupling mechanism is shaped to form an inner contour of the conductive material that receives the conductive electrode shaft and exerts a inward force on the conductive electrode shaft, an outer contour of the conductive material that exerts an outward force on the energy dissipating structure, and a relief segment somewhere along the coupling mechanism that is designed to be more susceptible to deformation due to forces than the rest of the coupling mechanism.

In another example, the disclosure is directed to a coupling mechanism formed of a conductive material. The conductive material of the coupling mechanism is shaped to form an inner contour of the conductive material that receives a first structure and exerts a inward force on the first structure, an outer contour of the conductive material that exerts an outward force on a second structure, and a relief segment somewhere along the coupling mechanism that is designed to be more susceptible to deformation due to forces than the rest of the coupling mechanism.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

FIG. 1 is a conceptual diagram illustrating an environment in which an implantable medical system is exposed to external fields.

FIG. 2 is a schematic diagram illustrating an example implantable medical system.

FIG. 3 is a schematic diagram illustrating a longitudinal cross-sectional view of a distal end of a lead.

FIGS. 4A-4C are schematic diagrams illustrating an example coupling mechanism from various viewpoints.

FIGS. 5A-5C are schematic diagrams illustrating another example coupling mechanism from various viewpoints.

FIGS. 6A-6C are schematic diagrams illustrating another example coupling mechanism from various viewpoints.

FIG. 7 is a schematic diagram illustrating a front view of another example coupling mechanism.

FIGS. 8A-8C are schematic diagrams illustrating another example coupling mechanism from various viewpoints.

FIG. 9A and 9B are schematic diagrams illustrating another example coupling mechanism from various viewpoints.

FIG. 10A and 10B are schematic diagrams illustrating another example coupling mechanism from various viewpoints.

FIG. 11 is a schematic diagram illustrating a front view of another example coupling mechanism.

### DETAILED DESCRIPTION

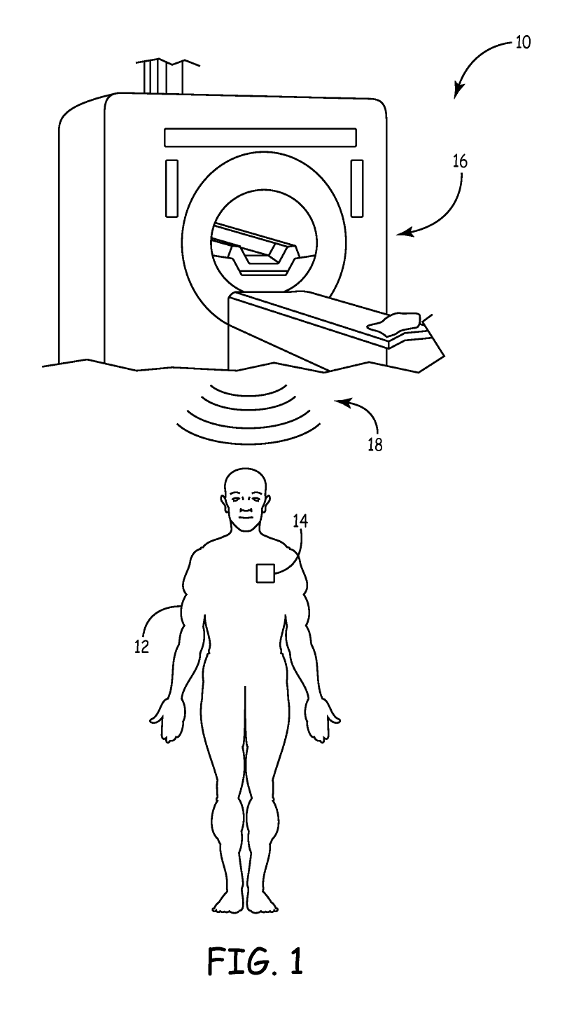

FIG. 1 is a conceptual diagram illustrating an environment 10 in which a patient 12 with an implantable medical system 14 is exposed to external fields 18. In the example illustrated in FIG. 1, environment 10 includes an MRI device 16 that generates external fields 18. MRI device 16 generates magnetic and RF fields to produce images of body structures for diagnosing injuries, diseases and/or disorders. In particular, MRI device 16 generates a static magnetic field, gradient magnetic fields and RF fields as is well known in the art. The static magnetic field is a non time-varying magnetic field that is typically always present around MRI device 16 whether or not an MRI procedure is in progress. Gradient magnetic fields are pulsed magnetic fields that are typically only present while the MRI procedure is in progress. RF fields are pulsed high frequency fields that are also typically only present while the MRI procedure is in progress.

The magnitude, frequency or other characteristic of the static magnetic field, gradient magnetic fields and RF fields may vary based on the type of MRI device producing the field or the type of MRI procedure being performed. For example, a 1.5 Tesls (T) MRI device will produce a static magnetic field of about 1.5 T and have a corresponding RF frequency of about 64 megahertz (MHz) while a 3.0 T MRI device will produce a static magnetic field of about 3.0 T and have a corresponding RF frequency of about 128 MHz. However, other MRI devices may generate fields of different strengths and/or frequencies.

Implantable medical system 14 may, in one example, include an implantable medical device (IMD) connected to one or more leads. The IMD may be an implantable cardiac device that senses electrical activity of a heart of patient 12 and/or provides electrical stimulation therapy to the heart of patient 12. For example, the IMD may be an implantable pacemaker, implantable cardioverter defibrillator (ICD), cardiac resynchronization therapy defibrillator (CRT-D), cardioverter device, or combinations thereof. The IMD may alternatively be a non-cardiac implantable device, such as an implantable neurostimulator or other device that provides electrical stimulation therapy.

Although environment 10 is described as including an MRI device 16 that generates external fields 18, environment 10 may include other sources of external fields 18, such as devices used for electrocautery procedures, diathermy procedures, ablation procedures, electrical therapy procedures, magnetic therapy procedures or the like. Moreover, environment 10 may include a non-medical source of external fields 18, such as an interrogation unit of a radio frequency (RF) security gate.

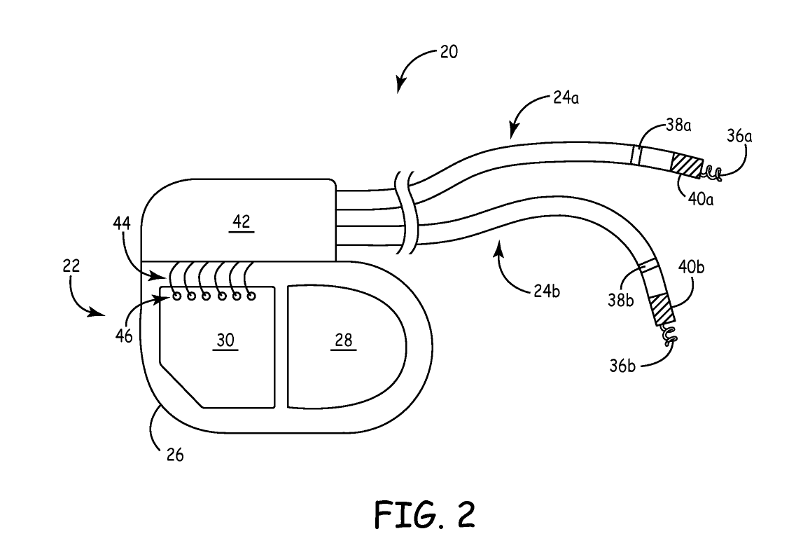

FIG. 2 is a schematic diagram illustrating an example implantable medical system 20. Implantable medical system 20 may, for example, correspond with implantable medical system 14 of FIG. 1. Implantable medical system 20 includes an IMD 22 and leads 24a and 24b (sometimes referred to herein as leads 24 or leads 24a,b). IMD 22 may be an implantable cardiac device that senses electrical activity of a heart and/or provides electrical stimulation therapy to the heart. IMD 22 may, for example, be an implantable pacemaker, implantable ICD, implantable CRT-D, implantable cardioverter device, or other device or combinations thereof. IMD 22 may alternatively be a non-cardiac implantable device, such as an implantable neurostimulator or other device that provides electrical stimulation therapy.

IMD 22 includes a housing 26 within which components of IMD 22 are housed. Housing 26 can be formed from conductive materials, non-conductive materials or a combination thereof. IMD 22 includes a power source 28 and a printed circuit board (PCB) 30 enclosed within housing 26. Power source 28 may include a battery, e.g., a rechargeable or non-rechargeable battery, or other power source. PCB 30 includes one or more electrical components (not shown in FIG. 2) of IMD 22, such as one or more processors, memories, transmitters, receivers, sensors, sensing circuitry, therapy circuitry and other appropriate components.

PCB 30 may provide electrical connections between power source 28 and the electrical components of IMD 22 such that power source 28 powers the various electrical components of PCB 30. In some examples, PCB 30 may include one or more layers of conductive traces and conductive vias that provide electrical connection between power source 28 and the electrical components as well as provide electrical connections among the various electrical components. PCB 30 may not be limited to typical PCB structures, but may instead represent any structure within IMD 22 that is used to mechanically support and electrically connect the electrical components of IMD 22 and power source 28. Moreover, although the electronics components of IMD 22 are described as being on a single PCB, it is contemplated that the electronic components described herein may be included elsewhere within IMD 22, e.g., on other supporting structures within IMD 22, such as additional PCBs (not shown).

Leads 24a,b each include a respective tip electrode 36a,b and ring electrode 38a,b located near a distal end of respective leads 24a,b. In other examples, however, leads 24a,b may include more or fewer electrodes. When implanted, tip electrodes 36a,b and/or ring electrodes 38a,b are placed relative to or in a selected tissue, muscle, nerve or other location. In the example illustrated in FIG. 2, tip electrodes 36a,b are extendable helically shaped electrodes to facilitate fixation of the distal end of leads 24a,b to the target location within patient 12. In this manner, tip electrodes 36a,b are formed to define a fixation mechanism. In other embodiments, one or both of tip electrodes 36a,b may be formed to define fixation mechanisms of other structures. In other instances, leads 24a,b may include a fixation mechanism separate from tip electrode 36a,b. In this case, tip electrodes 36a,b may be passive, such as a hemispherical electrode or ring electrode. Fixation mechanisms can be any appropriate type, including a grapple mechanism, a helical or screw mechanism, a drug-coated connection mechanism in which the drug(s) serves to reduce infection and/or swelling of the tissue, or other attachment mechanism.

Leads 24a,b are connected at a proximal end to IMD 22 via connector block 42. Connector block 42 may include one or more ports that interconnect with one or more connector terminals located on the proximal end of leads 24a,b. Leads 24a,b are ultimately electrically connected to one or more electrical components on PCB 30 through, for example, connecting wires 44 that may extend within connector block 42. For example, connecting wires 44 may be connected to leads 24a,b at one end, and connected to PCB connection points 46 on PCB 30 at the other end.

One or more conductors (not shown in FIG. 2) can extend within a body of leads 24a,b from connector block 42 to engage the ring electrode 38a,b and tip electrode 36a,b, respectively. The body of leads 24a,b may be formed from a non-conductive material, including silicone, polyurethane, fluoropolymers, mixtures thereof, and other appropriate materials, shaped to form a lumen within which the one or more conductors extend. In this manner, each of tip electrodes 36a,b and ring electrodes 38a,b is electrically coupled to a respective conductor within the lumen of the associated lead bodies. For example, a first electrical conductor can extend along the length of body of lead 24a from connector block 42 and electrically couple to tip electrode 36a and a second electrical conductor can extend along the length of the body of lead 24a from connector block 42 and electrically couple to ring electrode 38a. The respective conductors may couple to circuitry, such as a therapy module or a sensing module, of IMD 22 via connections in connector block 42, connecting wires 44 and PCB connection points 46. The electrical conductors transmit therapy from the therapy module within IMD 22 to combinations of electrodes 36a,b and 38a,b and transmit sensed electrical signals from electrodes 36a,b and 38a,b to the sensing module within IMD 22.

A patient having implanted medical system 20 may receive a certain therapy or diagnostic technique that exposes implantable medical system 20 to external fields, such as external fields 18 of FIG. 1. In the case of an MRI procedure, for example, implantable medical system 20 is exposed to high frequency RF pulses and various magnetic fields to create image data regarding the patient 12. The RF pulses can induce currents within the leads 24a,b of the IMD 22. The current induced in the leads 24a,b can cause certain effects, including heating, of the various lead components and/or tissue near the lead. According to various embodiments, such as those discussed herein, components or mechanisms can be provided to reduce or eliminate the amount of current at tip electrodes 36a,b and/or ring electrodes 38a,b.

According to various embodiments discussed herein, one or both of leads 24a,b include components or mechanisms to reduce or eliminate the amount of current induced by external fields. To this end, each of leads 24a,b includes a respective energy dissipating structure 40a,b to which at least a portion of the current induced on leads 24a,b is redirected. Redirecting or shunting at least a portion of the induced current from tip electrodes 36a,b to energy dissipating structures 40a,b increases the area over which the current or thermal energy is dissipated, thereby decreasing the amount of heating adjacent to tip electrodes 36a,b. Energy dissipating structures 40a,b may, for example, comprise a conductive housing, a ring electrode, a sheath, a sleeve head, or a thermally conductive element. In this manner, the medical electrical leads described in this disclosure may allow a patient to undergo medical procedures that utilize high frequency signals without significantly affecting operation of the implantable medical system.

The embodiments described in this disclosure are discussed in the context of reducing induced current to tip electrodes 36a,b. However, this disclosure is not limited to such embodiments. One of skill in the art would understand that modifications may be made to reduce the amount of induced current conducted to ring electrodes 38a,b in addition to or instead of tip electrodes 36a,b. As such, the configuration of implantable medical system 20 of FIG. 2 is merely an example. Modifications may be made while still remaining within the scope of this disclosure.

Although described in the context of a coaxial lead, the techniques of this disclosure may be used in the context of multi-lumen leads with conductors within one or more of the multiple lumens. In other examples, implantable medical system 20 may include more or fewer leads extending from IMD 22. For example, IMD 22 may be coupled to three leads, e.g., implanted within the right atrium, right ventricle and left ventricle of the heart. In another example, IMD 22 may be coupled to a single lead that is implanted within either an atrium or ventricle of the heart. As such, implantable medical system 20 may be used for single chamber or multi-chamber cardiac rhythm management therapy.

In addition to more or fewer leads, each of the leads 24 may include more or fewer electrodes. In instances in which IMD 22 is used for therapy other than pacing, e.g., defibrillation or cardioversion, the leads may include elongated electrodes, which may, in some instances, take the form of a coil. IMD 22 may deliver defibrillation or cardioversion shocks to the heart via any combination of the elongated electrodes and housing electrode. As another example, medical system 20 may include leads with a plurality of ring electrodes, e.g., as used in some implantable neurostimulator, with a conductor associated with each of the plurality of ring electrodes and having one or a plurality of lumens.

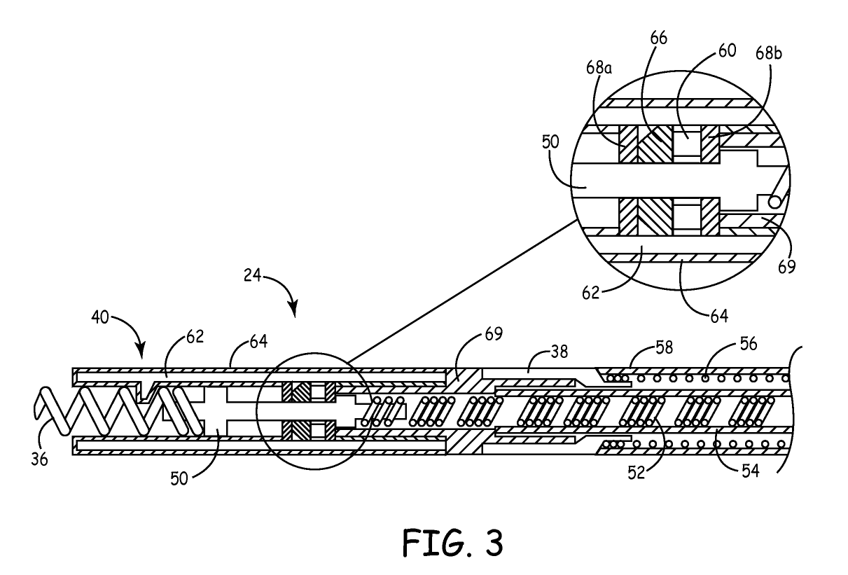

FIG. 3 is a schematic diagram illustrating a longitudinal cross-sectional view of a distal end of a lead 24. Lead 24 may correspond with lead 24a or lead 24b of FIG. 2. Lead 24 includes a tip electrode 36 and a ring electrode 38. Tip electrode 36 and ring electrode 38 may be electrically coupled to one or more electronic components on PCB 30 of IMD 22. As such, an electrical path exists from a proximal end of lead 24 (which is coupled to connector block 42 of IMD 22) to tip electrode 36 and ring electrode 38. A first electrical path may extend from IMD 22 through a conductor 52 and electrode shaft 50 to tip electrode 36. A second electrical path may extend from IMD 22 through a conductor 56 to ring electrode 38.

Ring conductor 56 is located within a body of lead 24 and extends along a length of lead 24 to electrically couple to ring electrode 38. Ring conductor 56 may be comprised of one or more conductive wires each surrounded by a respective insulating jacket. A proximal end of ring electrode 38 may be formed to receive a portion of ring conductor 56. Ring conductor 56 and ring electrode 38 are mechanically coupled (e.g., via welding, soldering, crimping or other mechanism). Ring electrode 38 is illustrated in FIG. 3 as having a cylindrical shape, but other shaped electrodes may be utilized in place of a ring electrode.

In the example illustrated in FIG. 3, electrode shaft 50 is connected at one end to tip conductor 52 and at the other end to tip electrode 36. Electrode shaft 50 may be connected to tip conductor 52 and tip electrode 36 via welding, soldering, crimping or other connection mechanism. Tip conductor 52, electrode shaft 50 and tip electrode 36 may all be formed at least partially from a conductive material, such as titanium, titanium alloy, tantalum, platinum, platinum iridium, conductive polymers, and/or other suitably conductive material or combination of materials. Tip conductor 52, electrode shaft 50 and tip electrode 36 may be all formed of the same conductive material or different conductive materials.

The mechanical coupling of tip conductor 52, electrode shaft 50 and tip electrode 36 provides a mechanical relationship that may, in some instances, allow for mechanical control of tip electrode 36 such that it may be extended from and retracted within the distal end of lead 24. During implantation, for example, a physician or other user may interact with lead 24 to rotate tip conductor 52, which causes electrode shaft 50 to rotate and extend tip electrode 36 from the distal end of lead 24. In this manner, tip electrode 36 may be screwed into the target tissue location. As such, tip conductor 52 may have sufficient rigidity to assist in attaching tip electrode 36 to the target tissue location while being flexible enough to navigate through body lumens, e.g., through one or more veins. In other examples, tip electrode 36 may be extended via a translational force instead of a rotational force. In other instances, electrode shaft 50 may be formed to receive a stylet to allow a user to extend and/or retract tip electrode 36. In further instances, lead 24 may not include an electrode shaft 50. Instead, conductor 52 may be directly connected to tip electrode 36, as in the case of a passive tip electrode that does not function as a fixation mechanism.

In the example illustrated in FIG. 3, ring conductor 56 is illustrated as having a larger diameter than tip conductor 52. In other instances, tip conductor 52 may have a larger diameter than ring conductor 56 or may have an equal diameter and run the length of lead 24 intertwined with ring conductor 56. In any case, at the proximal end of lead 24, tip conductor 52 and ring conductor 56 are electrically coupled to one or more electrical components IMD 22, such as an electrical stimulation module or sensing module, via connector block 42. Electrical stimulation may be delivered from IMD 22 to tip electrode 36 and/or ring electrode 38 and sensed electrical signals may be delivered from tip electrode 36 and/or ring electrode 38 via their respective conductors.

As described above, certain therapy or diagnostic techniques, such as an MRI procedure, may expose lead 24 to high frequency RF pulses and magnetic fields. The RF pulses can induce currents on conductors 52 or 56 within lead 24 of the IMD 22. The induced current on conductors 52 and 56 may be conducted to tip electrode 36 and ring electrode 38, respectively. Lead 24 includes components or mechanisms that can reduce the amount of induced current that is conducted to tip electrode 36. Although described in the context of reducing current to tip electrode 36, lead 24 may include similar mechanism or other mechanisms that may reduce the induced current conducted to ring electrodes 38 in addition to tip electrodes 36.

Lead 24 includes an energy dissipating structure 40 that is coupled to tip electrode 36. In some instances, energy dissipating structure 40 is electrically coupled to tip electrode 36. In other instances, energy dissipating structure 40 is non-conductively coupled, e.g., capacitively coupled or thermally coupled, to tip electrode 36. In the example illustrated in FIG. 3, energy dissipating structure 40 is coupled to tip electrode 36 through electrode shaft 50 and spring clip 60. In other words, spring clip 60 is the coupling mechanism that couples energy dissipating structure 40 to tip electrode 36. In other instances, spring clip 60 may provide contact between energy dissipating structure 40 and another portion of the electrical path to tip electrode 36, such as contact with the tip conductor 52 or direct contact with tip electrode 36.

As will be further described herein, spring clip 60 is formed to provide continuous contact with both electrode shaft 50 and energy dissipating structure 40. When assembled in the distal end of lead 24, spring clip 50 may be in a compressed state in which it provides forces in one or more radial directions from the longitudinal axis of electrode shaft 50. In particular, an inner contour of spring clip 60 exerts radial forces inward toward electrode shaft 50 and an outer contour of spring clip 60 exerts radial forces outward toward energy dissipating structure 40. The radial forces provide continuous contact with electrode shaft 50 and energy dissipating structure 40, thereby reducing the amount of noise that may be produced by intermittent contact between the conductive elements, e.g., due to polarization of the heart that causes movement of one conductive element relative to the other. However, spring clip 60 is designed such that forces on electrode shaft 50 are small enough to allow tip electrode 36 to be extended and retracted from lead 24. Thus, spring clip 60 allows electrode shaft 50 to move relative energy dissipating surface 40 while still maintaining continuous contact. Several examples of coupling mechanism 50 will be described in further detail herein.

In the example illustrated in FIGS. 2 and 3, energy dissipating structure 40 is a cylindrical ring. In other embodiments, however, energy dissipating structure 40 may be any other shape or geometry. In some instances, energy dissipating structure 40 may be made up of a plurality of surfaces. Energy dissipating structure 40 may be part of a housing, a ring electrode, a sheath, a sleeve head or other structure of lead 24. In this manner, energy dissipating structure 40 may provide a dual function, e.g., as a shunt and as part of a lead body or electrode housing. Energy dissipating structure 40 may be separated from ring electrode 38 via a non-conductive spacer 69.

As will be described in more detail herein, energy dissipating structure 40 presents a high impedance at low frequencies. As such, at low frequencies (e.g., ˜1 kHz for pacing signals), such as those used for pacing or other stimulation therapies, only a small amount of current is redirected to energy dissipating structure 40. In one example, less than approximately 5% of the current from low frequency pacing pulses are redirected to energy dissipating structure 40 and, in some instances, less than approximately 1%. However, the amount of current from low frequency signals that is redirected to energy dissipating structure 40 may be greater than or less than 5%, but still a small amount, e.g., less than 20% or, more preferably, less than 10%. At high frequencies, such as those produced during an MRI procedure, energy dissipating structure 40 presents a low impedance, resulting in a significant amount of the induced current being redirected to energy dissipating structure 40. In one example, at least approximately 80% of the current induced by the external field is redirected to energy dissipating structure 40, while less than approximately 20% of the induced current is conducted to tip electrode 36. However, the amount of current that is redirected away from tip electrode 36 may be smaller than or greater than 80%, e.g., approximately 50% or, more preferably, 60% or, even more preferably 70%. In this manner, the distal end of lead 24 is designed such that current from high frequency signals is redirected away from tip electrode 36 without significantly interfering with delivery of electrical stimulation therapy.

In some instances, energy dissipating structure 40 has a surface area that is significantly larger than a surface area of tip electrode 36. The surface area of energy dissipating structure 40 may, in one example, be between approximately 20-100 mm², which is at least approximately ten times larger than the surface area of tip electrode 36. A large surface area ratio, defined by the ratio of the surface area of energy dissipating structure 40 to the surface area of tip electrode 36 is desired to dissipate the induced current over a larger area to reduce heating at any specific location.

In the example illustrated in FIG. 3, energy dissipating structure 40 includes a conductive element 62 that is at least partially covered by a layer of insulating material 64. In other instances, however, conductive element 62 of energy dissipating structure 40 may be directly exposed to bodily fluid and/or tissue, i.e., not include the layer of insulating material 64. Conductive element 62 may be an electrically and thermally conductive element, such as titanium, titanium alloy, tantalum, platinum, platinum iridium, conductive polymers, and/or other suitably conductive material or combination of materials.

Insulating material 64 may cover an outer surface of conductive element 62 or at least a portion of the outer surface of conductive element 62. Insulating material 64 may affect the impedance of energy dissipating structure 40 and reduce the effect of energy dissipating structure 40 on the tip electrode to tissue interface impedances. As the thickness of insulating material 64 increases, the capacitance associated with energy dissipating structure 40 decreases and the impedance of energy dissipating structure 40 increases. As a result the amount of current redirected to energy dissipating structure 40 is reduced, but there is less interference with therapy delivered by IMD 22. As the thickness of insulating material 64 decreases, the capacitance associated with energy dissipating structure 40 increases and the impedance of energy dissipating structure 40 decreases. As a result the amount of current (even at low frequencies) redirected to energy dissipating structure 40 is increased, which may affect therapy delivered IMD 22.

For example, an energy dissipating surface 40 having a surface area of approximately 22 square millimeters (mm²) and an insulating material 64 having a dielectric constant of approximately 4.0, an insulating material thickness of approximately 68 micrometers provides an impedance of approximately 10 Ohms and a capacitance of approximately 250 pF, a thickness of approximately 34 micrometers provides an impedance of approximately 5 Ohms and a capacitance of approximately 500 pF, and a thickness of approximately 17 micrometers provides an impedance of approximately 2.5 Ohms and a capacitance of approximately 1 nF. These values are only exemplary in nature. The electrical characteristics of energy dissipating surface 40 may take on different values depending on the construction of the distal end of lead 24, e.g., based on the surface area of tip electrode 36, the surface area of energy dissipating surface 40, the construction of spring clip 60, the thickness of insulating material 64, the material from which the various components are constructed, and the like.

The thickness of insulating material 64 may be selected by a therapy system designer to achieve a satisfactory tradeoff between capacitance and impedance. Numerous techniques may be employed to introduce insulating material 64 over the outside of energy dissipating structure 40 and/or partially inside energy dissipating structure 40. Exemplary techniques include chemical vapor deposition, dip layer, spraying, thermal reflow, or thermal extrusion.

Insulating material 64 may also cover at least a portion of an inner surface of conductive element 62. Insulating material 64 on the inner surface may prevent conductive element 62 of energy dissipating structure 40 from making direct contact with the conductive material of tip electrode 36, electrode shaft 50 and/or tip conductor 52 at locations other than spring clip 60. In some instances, the insulating material 64 may even cover the surface of the portion of conductive element 62 that contacts spring clip 60. In this case, the coupling between spring clip 60 and conductive element 62 is a non-conductive (e.g., capacitive or thermal) coupling instead of a conductive coupling. In some instances, energy dissipating structure 40 may include more than one layer of insulating material, with each layer being made of the same or different insulating material. Insulating material 64 may include parylene, polyamide, metal oxides, polyimide, urethane, silicone, tetrafluroethylene (ETFE), polytetrafluroethylene (PTFE), polyether ether ketone (PEEK), oxides, or other suitable non-conductive material or combination of materials.

Lead 24 also includes a seal 66. Seal 66 is in contact with energy dissipating structure 40 and electrode shaft 50 to obtsruct fluid from passing into the lumen defined by the body of the lead. Seal 66 may be substantially ring (e.g. o-ring) or disk shaped but other suitable shapes may also be employed. In one example, seal 66 may be a non-conductive sealing washer or a conductive sealing washer with a non-conductive coating. Lead 24 may also include one more rings 68a,b that may hold seal 66 in place. In some instances, energy dissipating structure 40 and/or electrode shaft 50 may also be in contact with rings 44. Rings 44 may, in one example, be shaped as a non-conductive C-ring to receive seal 66. However, rings of other shapes may be used. In other instances, lead 24 may include only one ring 68a and spring clip 60 may function as both the coupling mechanism to energy dissipating structure 40 and as a mechanism to hold seal 66 in place. In other words, spring clip 50 may serve to function in the same manner as ring 68b in addition to a coupling mechanism to energy dissipating structure 40. In further instances, lead 24 may not include any of rings 68a,b. In this case, another spring clip 50 may be used on the opposite side of seal 66 such that energy dissipating structure 40 is coupled to electrode shaft 50 by two spring clips 50.

Lead 24 of FIG. 3 is one example of an electrode assembly in accordance with this disclosure. Modifications may be made while still remaining within the scope of this disclosure. For example, instead of a helical tip electrode, tip electrode 36 may take the form of a ring electrode, hemispherical electrode or other electrode. As another example, spring clip 50 may located elsewhere along the distal end of the lead, e.g., at locations away from seal 66 or rings 68a,b. Other modifications are also within the scope of this disclosure.

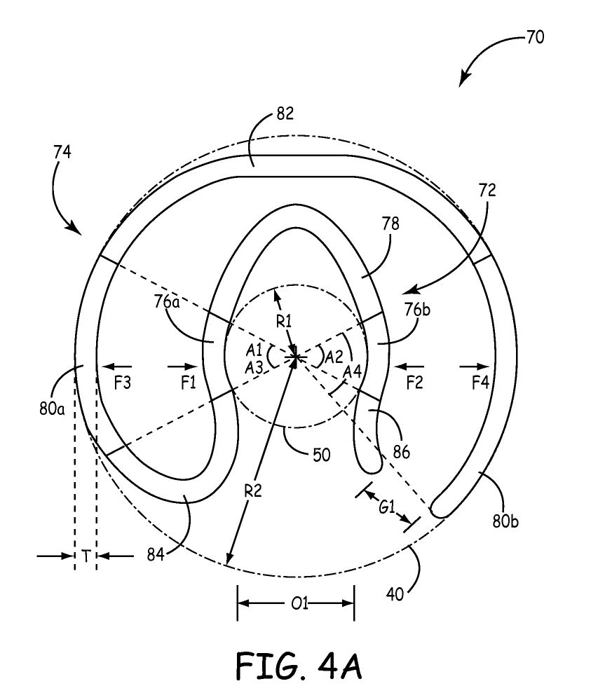

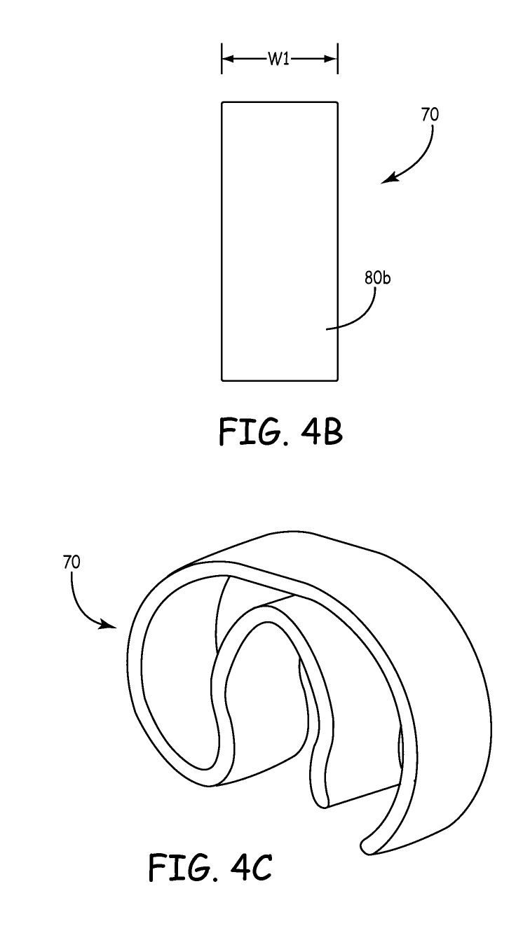

FIGS. 4A-4C are schematic diagrams illustrating an example coupling mechanism, e.g., spring clip 70, from various viewpoints. FIG. 4A is a front view of spring clip 70, FIG. 4B is a side view of spring clip 70 and FIG. 4C is an angled view of spring clip 70. Spring clip 70 may correspond with spring clip 60 of FIG. 3. Spring clip 70 is configured to provide continuous contact between electrode shaft 50 and energy dissipating structure 40 in an assembled lead. To this end, spring clip 70 includes an inner contour 72 configured to provide contact with electrode shaft 50 and an outer contour 74 configured to provide contact with conductive element 62 of energy dissipating structure 40.

In the example illustrated in FIGS. 4A-4C, inner contour 72 is located within the area defined by outer contour 74. In particular, inner contour 72 is substantially coplanar with outer contour 74, i.e., inner contour 72 lies in the same plane as outer contour 74. In such an arrangement space exists between the portion of the conductor forming the outer surface of inner contour 72 and the portion of the conductor forming the inner surface of outer contour 74. In addition to being coplanar, inner contour 72 and outer contour 74 may be substantially coaxial. In other instances, however, inner contour 72 may lie within a different plane than outer contour 74, i.e., non-coplanar, as described in other examples herein, and/or non-coaxial.

Inner contour 32 and outer contour 34 may be formed in any of a variety of geometries. Moreover, inner contour 32 and outer contour 34 may be formed in different geometries. Spring clip 70 is described below in terms of “segments” for purposes of description. The various segments may not actually be separate segments that are interconnected. Instead, spring clip 70 may be made of one continuous piece of conductive material.

Inner contour 72 of FIGS. 4A-4C, for example, includes contact segments 76a,b that provide continuous contact to electrode shaft 50 in an assembled lead. Contact segment 76a extends along an arc of a circle having a radius R1 and a central angle A1. Contact segment 76b extends along an arc of the circle having a radius R1 and a central angle A2. In some instances, central angle A1 and A2 may be equal. In other instances central angle A1 and A2 may be different. In one example, the radius R1 of contact segments 76a,b is equal to the radius of electrode shaft 50 when spring clip 70 is in a relaxed state (e.g., prior to being inserted within lead 24). In another example, the radius R1 of contact segments 76a,b is slightly smaller than the radius of electrode shaft 50 when spring clip 70 is in the relaxed state. Contact segments 76a,b make continuous contact with electrode shaft 50 along at least a portion of the contact segments 76a,b when inserted within lead 24. Contact segments 76a,b are connected by a curved segment 78. Curved segment 78 may be shaped like a parabola, a segment of an ellipse, a bullet-nose curve, an elliptical curve or other shape.

Outer contour 74 of spring clip 70 includes contact segments 80a,b that provide continuous contact to energy dissipating structure 40 in an assembled lead. Contact segment 80a extends along an arc of a circle having a radius R2 and a central angle A3. Contact segment 80b extends along an arc of the circle having a radius R2 and a central angle A4. In some instances, central angle A3 and A4 may be different. In other instances, central angle A3 and A4 may be equal. In one example, the radius R2 of contact segments 80a,b is slightly smaller than the radius of the energy dissipating structure 40 when spring clip 70 is in the relaxed state. In another example, the radius R2 of contact segments 80a,b is slightly smaller than the radius of energy dissipating structure 40 when spring clip 70 is in the relaxed state. Contact segments 80a,b make contact with energy dissipating structure 40 along at least a portion of the contact segments 80a,b in an assembled lead. Contact segments 80a,b are connected by a segment 82. Segment 82 may be made of one or more curved segments and/or one or more straight segments.

Spring clip 70 includes a curved segment 84 that connects contact segment 76a of inner contour 72 to contact segment 80a of outer contour 74. Spring clip 70 also includes a curved segment 86 that extends from contact segment 76b to help hold electrode shaft 50 within inner contour 72. Segment 86 of inner contour 72 does not, however, contact segment 80b of outer contour 74. Instead, a gap (G1) exists between the open end of segment 86 and the open end of segment 80b. In other words, there is a break along the transition from inner contour 72 to outer contour 74 such that the open end of segment 86 and the open end of segment 80b can move with respect to one another. The length of gap G1 may vary widely as long as open ends of segment 86 and segment 80b can move with respect to one another.

Again, spring clip 70 is described as being made up of a number of “segments” for purposes of illustration. However, spring clip 70 may be made of one continuous piece of conductive material that extends from the open end of segment 86, around inner contour 72, and around outer contour 74 to the open end of segment 80b. Moreover, spring clip 70 may have more or fewer segments and the segments may have different geometries. The segments may be different curved shapes, straight line segments, or any other shape.

Spring clip 70 is formed to include an opening (O1) via which electrode shaft 50 may be inserted within spring clip 70. In the example illustrated in FIG. 4A, the inner contour and the outer contour of the conductive material extend around less than an entire circumference of the respective contours such that opening O1 is formed via which the electrode shaft received. As will be described in further detail herein, spring clip 70 may deform elastically (e.g., opening O1 may become larger) during insertion of electrode shaft 50 into inner contour 72 of spring clip 70 and return to substantially the original shape (e.g., opening O1 returns to substantially its original size) after electrode shaft is inserted within inner contour 72 of spring clip 70. In some instances, spring clip 70 may remain slightly elastically deformed after insertion to provide the inward radial forces that provide continuous contact with electrode shaft 50. In other words, the radius of the outer surface of electrode shaft 50 may be approximately equal to or slightly larger than the radius (e.g., R1) of the inner surface of contact segments 76a,b. Opening O1 makes assembly of the distal end of lead 24 much easier by allowing insertion of electrode shaft 50 into inner contour 72 of spring clip 70 from the side instead of the coupling mechanism being slid on from one of the ends of the electrode shaft, as is the case for coupling mechanism having outer contours that extend around an entire circumference with no opening along the side.

After being attached to electrode shaft 50, energy dissipating structure 40 may be closed around spring clip 70. The interior radius of energy dissipating structure 40 may be approximately equal to or slightly smaller than the radius (e.g., R2) of the outer surface of contact segments 80a,b. As a result, the force applied to spring clip 70 by energy dissipating structure 40 may result in a slight elastic deformation of spring clip 70. By designing spring clip 70 to have gap G1, some of the force placed on the segments of spring clip 70 is relieved by the change in shape of spring clip 70 allowed by gap G1. In addition to changes to the size of gap G1, the size of opening O1 may also slightly change due to the deformation of spring clip 70. In this manner, relief of the force placed on the segments of sprig clip 70 may be from combination of changes to gap G1 and opening O1.

When assembled into a lead, spring clip 70 is in a “compressed” state that provides continuous contact with both electrode shaft 50 and energy dissipating structure 40. In the compressed state, spring clip 70 exerts an inward contact force toward electrode shaft 50 along at least a portion of contact segments 76a,b of inner contour 72 (represented as forces F1 and F2) and exerts an outward contact force toward energy dissipating structure 40 along at least a portion of contact segments 80a,b of outer contour 74 (represented as forces F3 and F4). The forces exerted by inner contour 72 on electrode shaft 50 (e.g., forces F1 and F2) should be large enough to provide for continuous contact with electrode shaft 50 even when tip electrode 36 is extended or retracted from the lead. However, forces F1 and F2 should be small enough such that electrode shaft 50 may be moved to extend or retract electrode 36. In other words, the forces should be small enough to permit rotational, translational, or other movement to extend and/or retract tip electrode 36. In one example, forces F1 and F2 should create a torque that is less than or equal to approximately 0.15 inch-ounces. In another example, forces F1 and F2 should create a torque that is less than or equal to approximately 0.03 inch-ounces. In a further example, forces F1 and F2 should create a torque that is less than or equal to approximately 0.003 inch-ounces. The forces exerted by outer contour 74 on energy dissipating structure 40 (e.g., forces F3 and F4) should be large enough to provide for continuous, stationary contact with energy dissipating shunt 40.

A number of characteristics of spring clip 70 determine the size of forces F1-F4 and the amount of stress and strain placed on portions of spring clip 70. Some example characteristics that influence force, stress and/or strain include the dimensions of spring clip 70, the shape of spring clip 70, the location of gap G1, the size of gap G1, the amount of surface area over which contact with inner contour 72 and outer contour 74 is exerted, the type of material used to form spring clip 70 and the like. These characteristics may be adjusted to achieve a desired force, strain distribution and stress distribution.

Spring clip 70 may be constructed from a conductor having a substantially round geometry, a substantially flat geometry or other geometry. In the example illustrated in FIGS. 4A-4C, spring clip 70 is constructed of a conductor having a substantially flat geometry. The conductor may, in one example, have a width (W) of approximately 0.02 to 0.04 inches. However, the conductor can have larger or smaller widths. The conductor may be made of a number of different materials, including platinum, platinum iridium (Pt/Ir), nickel-cobalt based alloy, titanium, tantalum, platinum clad tantalum, stainless steel, silver cored nickel-cobalt based alloy, silver cored tantalum, and niobium or other conductive material or combination or conductive materials. The conductive material of spring clip 70 may have a thickness (illustrated in FIG. 4A and labeled “T”) of less than approximately 5 mil. In some instances, thickness T may be less than or equal to approximately 3 mil. Typically, a smaller thickness T results in smaller radial contact forces and less stress and strain placed on the various segments of spring clip 70.

In some instances, the conductor forming spring clip 70 may include a layer of non-conductive material or capacitive material or inductive material (not shown in FIGS. 4A-4C) that covers at least a portion of the conductive material of the coupling mechanism. The layer of capacitive and/or inductive material may include one or more of parylene, polyamide, metal oxides, polyimide, urethane, silicone, tetrafluroethylene (ETFE), polyether ether ketone (PEEK), polytetrafluroethylene (PTFE), or the like. The insulating material may be thin, e.g., less than approximately 2 mil.

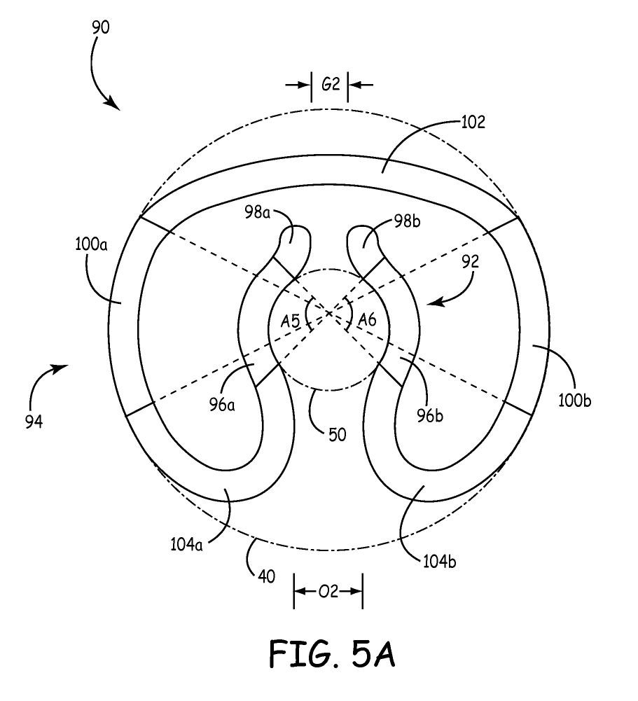

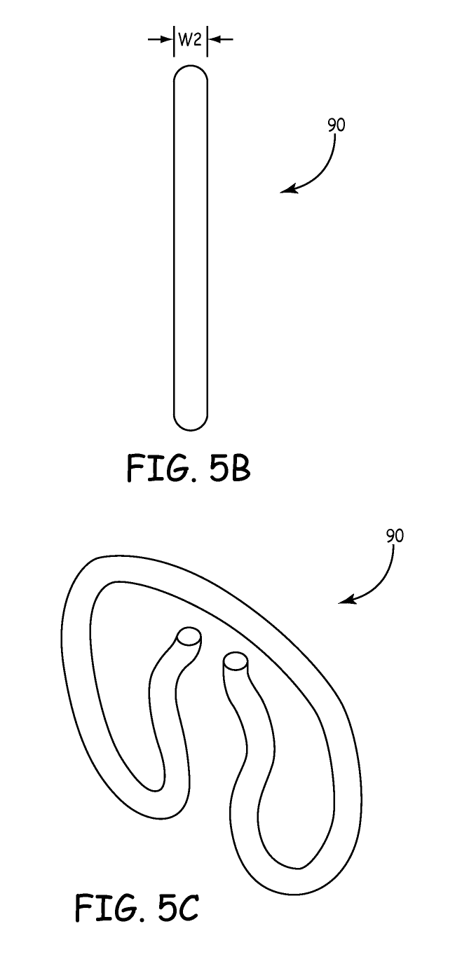

FIGS. 5A-5C are schematic diagrams illustrating another example spring clip 90 from various viewpoints. FIG. 5A is a front view of spring clip 90, FIG. 5B is a side view of spring clip 90 and FIG. 5C is an angled view of spring clip 90. Spring clip 90 may correspond with spring clip 60 of FIG. 3. Spring clip 90 includes an inner contour 92 configured to provide continuous contact with electrode shaft 50 and an outer contour 94 configured to provide continuous contact with conductive element 62 of energy dissipating structure 40. In some instances, inner contour 92 may be substantially coplanar and/or coaxial with outer contour 94.

Inner contour 92 includes contact segments 96a,b that provide continuous contact to electrode shaft 50 in an assembled lead. Contact segment 96a extends along an arc of a circle having a radius R1 and a central angle A5. Contact segment 96b extends along an arc of the circle having a radius R1 and a central angle A6. In some instances, central angle A5 and A6 may be equal. In other instances central angle A5 and A6 may be different. In the example illustrated in FIG. 5A, central angles A5 and A6 are larger than central angles A1 and A2 of contact segments 76a,b of FIG. 4A. In other words, a larger portion of contact segments 96a,b contacts electrode shaft 50 than contact segments 76a,b. In one example, the radius R1 of contact segments 96a,b is equal to the radius of electrode shaft 50 when spring clip 90 is in a relaxed state (e.g., prior to being inserted within lead 24). In another example, the radius R1 of contact segments 96a,b is slightly smaller than the radius of electrode shaft 50 when spring clip 90 is in the relaxed state.

Unlike contact segments 76a,b of FIG. 4A, contact segments 96a,b do not contact one another. Instead, short segments 98a,b extend from contact segments 96a,b to help hold electrode shaft 50 within inner contour 92. Segments 98a,b are each open at the end to form a gap G2. Thus, gap G2 is the space between the open end of segment 98a and the open end of segment 98b. In other words, there is a break along inner contour 92 such that the open end of segment 98a and the open end of segment 98b can move with respect to one another. The length of gap G2 may vary widely as long as open ends of segments 98a,b can move with respect to one another.

As will be described in more detail below, forces placed on the segments of spring clip 90 may be at least partially relieved by the deformation of spring clip 90 allowed by gap G2. In other words, gap G2 may become larger or smaller to relieve some of the forces on segments of spring clip 90. The location of gap G2 in FIGS. 4A-4C is for example purposes only. Inner contour 112 may be designed such that gap G2 is located anywhere along inner contour 112.

Outer contour 94 of spring clip 90 includes contact segments 100a,b that provide continuous contact to energy dissipating structure 40 in an assembled lead. Contact segment 100a extends along an arc of a circle having a radius R2 and a central angle A7. Contact segment 100b extends along an arc of the circle having a radius R2 and a central angle A8. In some instances, central angle A7 and A8 may be different. In other instances, central angle A7 and A8 may be equal. In one example, the radius R2 of contact segments 100a,b is slightly smaller than the radius of the energy dissipating structure 40 when spring clip 90 is in the relaxed state. In another example, the radius R2 of contact segments 100a,b is slightly smaller than the radius of energy dissipating structure 40 when spring clip 90 is in the relaxed state. Contact segments 100a,b make contact with energy dissipating structure 40 along at least a portion of the contact segments 100a,b in an assembled lead. Contact segments 100a,b are connected by a segment 102. Segment 102 may be made of one or more curved segments and/or one or more straight segments.

Inner contour 92 is connected to outer contour 94 on both sides. In particular, spring clip 90 includes a curved segment 104a that connects contact segment 96a of inner contour 92 to contact segment 100a of outer contour 94 and a curved segment 104b that connects contact segment 96b of inner contour 92 to contact segment 100b of outer contour 94. Although spring clip 90 is described as being made up of a number of “segments,” the various segments may not actually be separate segments that are interconnected. Instead, spring clip 90 may be made of one continuous piece of conductive material that extends from the open end of segment 98a around the inner contour and outer contour to the open end of segment 98b. Moreover, spring clip 90 may have more or fewer segments and the segments may have different shapes. The segments may be different curved shapes, straight line segments, or any other shape. In one example, segments 98a,b may extend further or include a straight segment. In another example, segments 98a,b may not even exist. Instead there may be a gap between contact segments 96a,b.

Spring clip 90 is formed to include an opening (O2) via which electrode shaft 50 may be inserted within spring clip 90. As will be described in further detail herein, spring clip 90 may deform elastically (e.g., opening O2 may become larger) during insertion of electrode shaft 50 into inner contour 92 of spring clip 90 and return to substantially the original shape (e.g., opening O2 returns to substantially its original size) after electrode shaft is inserted within inner contour 92 of spring clip 90. In some instances, spring clip 90 may remain slightly elastically deformed after insertion to provide the inward radial forces that provide continuous contact with electrode shaft 50. In other words, the radius of the outer surface of electrode shaft 50 may be approximately equal to or slightly larger than the radius (e.g., R1) of the inner surface of contact segments 96a,b. Opening O2 makes assembly of the distal end of lead 24 much easier by allowing insertion electrode shaft 50 into inner contour 92 of spring clip 90 from the side instead of the coupling mechanism being slid on from one of the ends of the electrode shaft, as is the case for coupling mechanism having outer contours that extend around an entire circumference with no opening along the side.

After being attached to electrode shaft 50, energy dissipating structure 40 may be closed around spring clip 90. The interior radius of energy dissipating structure 40 may be approximately equal to or slightly smaller than the radius (e.g., R2) of the outer surface of contact segments 100a,b. As a result, the pressure applied to spring clip 90 by energy dissipating structure 40 may result in a slight elastic deformation of spring clip 90. By designing spring clip 90 to have gap G2, some of the stress placed on the segments of spring clip 90 is relieved by the change in shape of spring clip 90 allowed by gap G2. In addition to changes to the size of gap G2, the size of opening O2 may also slightly change due to the deformation of spring clip 90. In this manner, relief of the stress placed on the segments of sprig clip 90 may be from combination of changes to gap G2 and opening O2.

When assembled into a lead, spring clip 90 is in a “compressed” state that provides continuous contact with both electrode shaft 50 and energy dissipating structure 40. In the compressed state, spring clip 90 exerts an inward contact force toward electrode shaft 50 along at least a portion of contact segments 96a,b of inner contour 92 (represented as forces F1 and F2) and exerts an outward contact force toward energy dissipating structure 40 along at least a portion of contact segments 100a,b of outer contour 94 (represented as forces F3 and F4). The forces exerted by inner contour 92 on electrode shaft 50 (e.g., forces F1 and F2) should be large enough to provide for continuous contact with electrode shaft 50 even when the tip electrode 36 is extended or retracted from the lead. However, forces F1 and F2 should be small enough to allow the tip electrode 36 to be extended and retracted from the lead. In one example, forces F1 and F2 should create a torque that is less than or equal to approximately 0.15 inch-ounces. In another example, forces F1 and F2 should create a torque that is less than or equal to approximately 0.03 inch-ounces. In a further example, forces Fl and F2 should create a torque that is less than or equal to approximately 0.003 inch-ounces. The forces exerted by outer contour 94 on energy dissipating structure 40 (e.g., forces F3 and F4) should be large enough to provide for continuous, stationary contact with energy dissipating shunt 40.

A number of characteristics of spring clip 90 determine the size of forces F1-F4 and the amount of stress and strain placed on portions of spring clip 90. Some example characteristics that influence force, stress and strain include the dimensions of spring clip 90, the shape of spring clip 90, the location of gap G2, the size of gap G2, the amount of surface area over which contact with inner contour 92 and outer contour 94 is exerted, the type of material used to form spring clip 90 and the like. These characteristics may be adjusted to achieve a desired force, strain distribution and stress distribution.

Spring clip 90 may be constructed from a conductor having a substantially round geometry, a substantially flat geometry or other geometry. In the example illustrated in FIGS. 5A-5C, spring clip 90 is constructed of conductor having a substantially round geometry. The wire may be made of a number of different materials, including platinum, platinum iridium (Pt/Ir), nickel-cobalt based alloy, titanium, tantalum, platinum clad tantalum, stainless steel, silver cored nickel-cobalt based alloy, silver cored tantalum, and niobium or other conductive material or combination or conductive materials. The conductive material of spring clip 90 may have a thickness (illustrated in FIG. 5A and labeled “T”) of less than approximately 5 mil. In some instances, thickness T may be less than or equal to approximately 3 mil. Typically, a smaller thickness T results in smaller radial contact forces and less stress and strain placed on the various segments of spring clip 90.

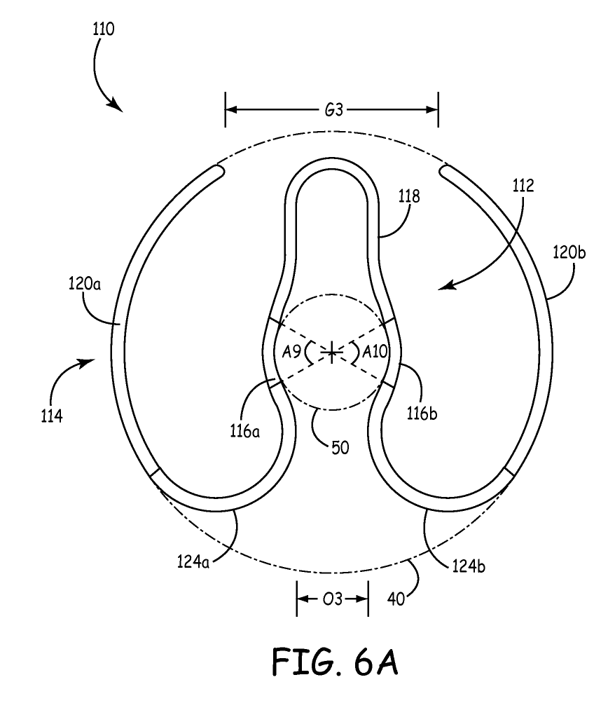

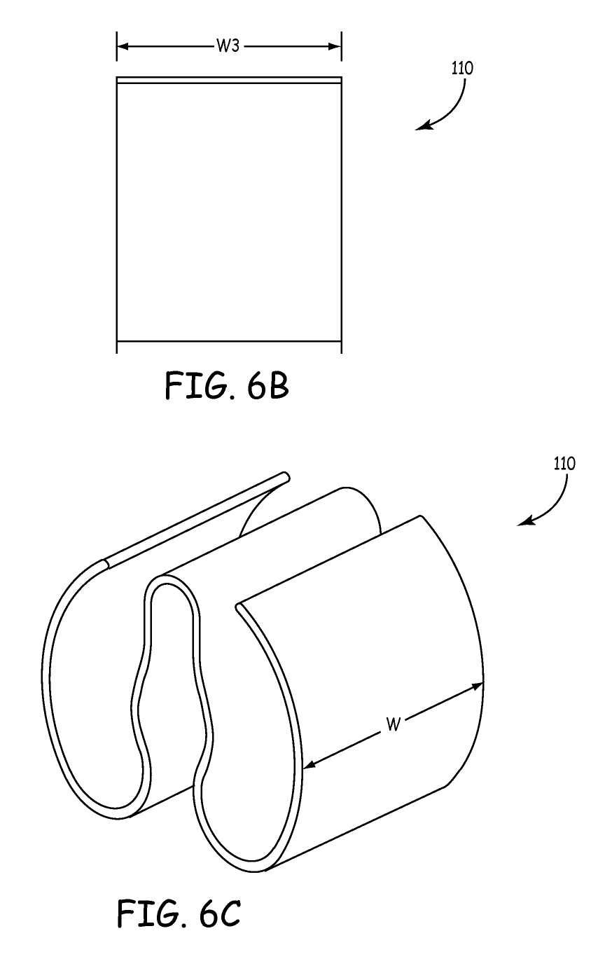

FIGS. 6A-6C are schematic diagrams illustrating an example spring clip 110 from various viewpoints. FIG. 6A is a front view of spring clip 110, FIG. 6B is a side view of spring clip 110 and FIG. 6C is an angled view of spring clip 110. Spring clip 110 may correspond with spring clip 60 of FIG. 3. Spring clip 110 includes an inner contour 112 configured to provide continuous contact with electrode shaft 50 and an outer contour 114 configured to provide continuous contact with conductive element 62 of energy dissipating structure 40 in an assembled lead. In some instances, inner contour 112 may be substantially coplanar and/or coaxial with outer contour 114.

Inner contour 112 includes contact segments 116a,b that provide continuous contact to electrode shaft 50 in an assembled lead. Contact segment 116a extends along an arc of a circle having a radius R1 and a central angle A9. Contact segment 116b extends along an arc of the circle having a radius R1 and a central angle A10. In some instances, central angle A9 and A10 may be equal. In other instances central angle A9 and A10 may be different. In one example, the radius R1 of contact segments 116a,b is equal to the radius of electrode shaft 50 when spring clip 110 is in a relaxed state (e.g., prior to being inserted within lead 24). In another example, the radius R1 of contact segments 116a,b is slightly smaller than the radius of electrode shaft 50 when spring clip 110 is in the relaxed state. Contact segments 116a,b make continuous contact with electrode shaft 50 along at least a portion of the contact segments 116a,b when inserted within the lead. Contact segments 116a,b are connected by a curved segment 118. Curved segment 118 may be shaped like a parabola, a segment of an ellipse, a bullet-nose curve, an elliptical curve or other shape. In other examples, segments 116a,b and 118 may have different shapes.

Outer contour 114 of spring clip 110 includes contact segments 120a,b that provide continuous contact to energy dissipating structure 40 in an assembled lead. Contact segment 120a extends along an arc of a circle having a radius R2 and a central angle A11. Contact segment 120b extends along an arc of the circle having a radius R2 and a central angle A12. In some instances, central angle A11 and A12 may be different. In other instances, central angle A11 and A12 may be equal. In one example, the radius R2 of contact segments 80a,b is slightly smaller than the radius of the energy dissipating structure 40 when spring clip 110 is in the relaxed state. In another example, the radius R2 of contact segments 80a,b is slightly smaller than the radius of energy dissipating structure 40 when spring clip 110 is in the relaxed state is equal to the radius of energy dissipating structure 40. Spring clip 110 includes curved segments 124a,b that connect respective contact segments 116a,b of inner contour 112 to respective contact segment 120a,b of outer contour 114. Therefore, inner contour 112 is connected to outer contour 114 on both sides.

Contact segments 120a,b are each open at one end such that there is a gap (G3) between the open end of contact segment 120a and the open end of contact segment 120b. In other words, there is a break along outer contour 74 such that the open end of segment 120a and the open end of segment 120b can move with respect to one another. The length of gap G3 may vary widely as long as open ends of segments 120a,b can move with respect to one another.

As will be described in more detail below, force placed on the segments of spring clip 110 may be at least partially relieved by deformation of spring clip 110 allowed by gap G3. In other words, gap G3 may become larger or smaller to relieve some of the forces on segments of spring clip 110. In the example illustrated in FIGS. 6A-6C, gap G3 is located on outer contour 114 adjacent to the segment 118 of inner contour. However, outer contour 114 may be designed such that gap G3 is located anywhere along outer contour 114.

Spring clip 110 is described as being made up of a number of “segments” for purposes of illustration. The various segments do may not actually have to be separate segments that are interconnected. Instead, spring clip 110 may be made of one continuous piece of conductive material that extends from the open end of segment 126 and extending around the inner contour and outer contour to the open end of segment 120b. Moreover, spring clip 110 may have more or fewer segments and the segments may have different shapes. The segments may be different curved shapes, straight line segments, or any other shape.

Spring clip 110 is formed to include an opening (O3) via which electrode shaft 50 may be inserted within spring clip 110. As will be described in further detail herein, spring clip 110 may deform elastically (e.g., opening O3 may become larger) during insertion of electrode shaft 50 into inner contour 112 of spring clip 110 and return to substantially the original shape (e.g., opening O3 returns to substantially its original size) after electrode shaft is inserted within inner contour 112 of spring clip 110. In some instances, spring clip 110 may remain slightly elastically deformed after insertion to provide the inward radial forces to provide continuous contact with electrode shaft 50. In other words, the radius of the outer surface of electrode shaft 50 may be approximately equal to or slightly larger than the radius (e.g., R1) of the inner surface of contact segments 116a,b. Opening O3 makes assembly of the distal end of lead 24 much easier by allowing insertion of electrode shaft 50 into inner contour 112 of spring clip 110 from the side instead of the coupling mechanism being slid on from one of the ends of the electrode shaft, as is the case for coupling mechanism having outer contours that extend around an entire circumference with no opening along the side.

After being attached to electrode shaft 50, energy dissipating structure 40 may be closed around spring clip 110. The interior radius of energy dissipating structure 40 may be approximately equal to or slightly smaller than the radius (e.g., R2) of the outer surface of contact segments 120a,b. As a result, the pressure applied to spring clip 110 by energy dissipating structure 40 may result in a slight elastic deformation of spring clip 110. By designing spring clip 110 to have gap G3, some of the stress placed on the segments of spring clip 110 is relieved by the change in shape of spring clip 110 allowed by gap G3. In addition to changes to the size of gap G3, the size of opening O3 may also slightly change due to the deformation of spring clip 110. In this manner, relief of the stress placed on the segments of sprig clip 110 may be from combination of changes to gap G3 and opening O3.

When assembled into a lead, spring clip 110 is in a “compressed” state that provides continuous contact with both electrode shaft 50 and energy dissipating structure 40. In the compressed state, spring clip 110 exerts an inward force toward electrode shaft 50 along at least a portion of contact segments 116a,b of inner contour 112 (represented as forces F1 and F2) and exerts an outward force toward energy dissipating structure 40 along at least a portion of contact segments 120a,b of outer contour 114 (represented as forces F3 and F4). The forces exerted by inner contour 112 on electrode shaft 50 (e.g., forces F1 and F2) should be large enough to provide for continuous contact with electrode shaft 50 even when tip electrode 36 is extended or retracted from the lead. However, forces F1 and F2 should be small enough to allow tip electrode 36 to be extended and retracted from the lead. In one example, forces F1 and F2 should create a torque that is less than or equal to approximately 0.15 inch-ounces. In another example, forces F1 and F2 should create a torque that is less than or equal to approximately 0.03 inch-ounces. In a further example, forces F1 and F2 should create a torque that is less than or equal to approximately 0.003 inch-ounces. The forces exerted by outer contour 114 on energy dissipating structure 40 (e.g., forces F3 and F4) should be large enough to provide for continuous, stationary contact with energy dissipating shunt 40.

A number of characteristics of spring clip 110 determine the size of forces F1-F4 and the amount of stress and strain placed on portions of spring clip 110. Some example characteristics that influence force, stress and strain include the dimensions of spring clip 110, the shape of spring clip 110, the location of gap G3, the size of gap G3, the amount of surface area over which contact with inner contour 112 and outer contour 114 is exerted, the type of material used to form spring clip 110 and the like. These characteristics may be adjusted to achieve a desired force, strain distribution and stress distribution.

Spring clip 110 may be constructed from a conductor having a substantially round geometry, a substantially flat geometry or other geometry. In the example illustrated in FIGS. 6A-6C, spring clip 90 is constructed from a conductor having a substantially flat geometry. The wire may be made of a number of different materials, including platinum, platinum iridium (Pt/Ir), nickel-cobalt based alloy, titanium, tantalum, platinum clad tantalum, stainless steel, silver cored nickel-cobalt based alloy, silver cored tantalum, and niobium or other conductive material or combination or conductive materials. The conductive material of spring clip 110 may have a thickness (illustrated in FIG. 6A and labeled “T”) of less than approximately 5 mil. In some instances, thickness T may be less than or equal to approximately 3 mil. Typically, a smaller thickness T results in smaller radial contact forces and less stress and strain placed on the various segments of spring clip 110.

In some instances, the length of segments 120a and 120b may extend further such that the open ends of segments 120a,b are adjacent to one another. In this case, segments 120a,b may overlap in the compressed state. In other words, segment 120a may fold under segment 120b when compressed by the forces.

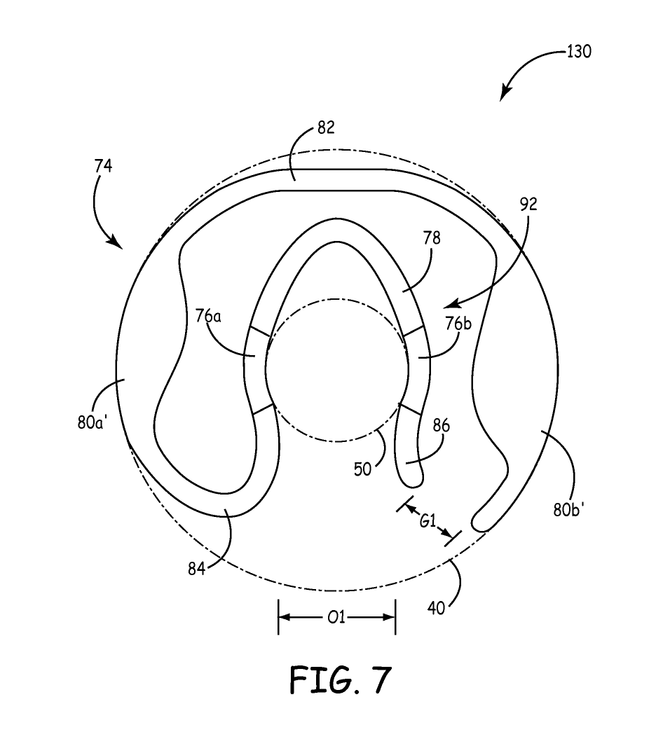

FIG. 7 is a schematic diagram illustrating a front view of another example spring clip 130. Spring clip 130 conforms substantially to spring clip 70 of FIGS. 4A and 4B except that spring clip 130 has a variable thickness. In the example illustrated in FIG. 7, contact segments 80a′ and 80b′ have an increased thickness along at least a portion of segments 80a′ and 80b′. The increased thickness of segments 80a′ and 80b′ provide additional surface area that may improve support of seal 66. In some instances, this may eliminate the need for at least one of rings 68a,b. The example illustrated in FIG. 7 shows segments 80a′ and 80b′ having the increased thickness and increase surface area. However, other segments of spring clip 130 may have the increased thickness instead of or in addition to segments 80a′ and 80b′.

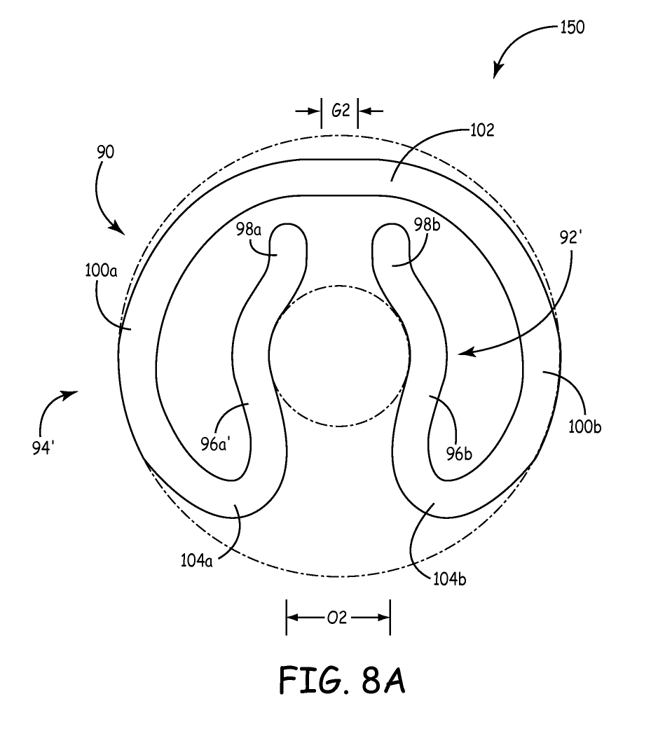

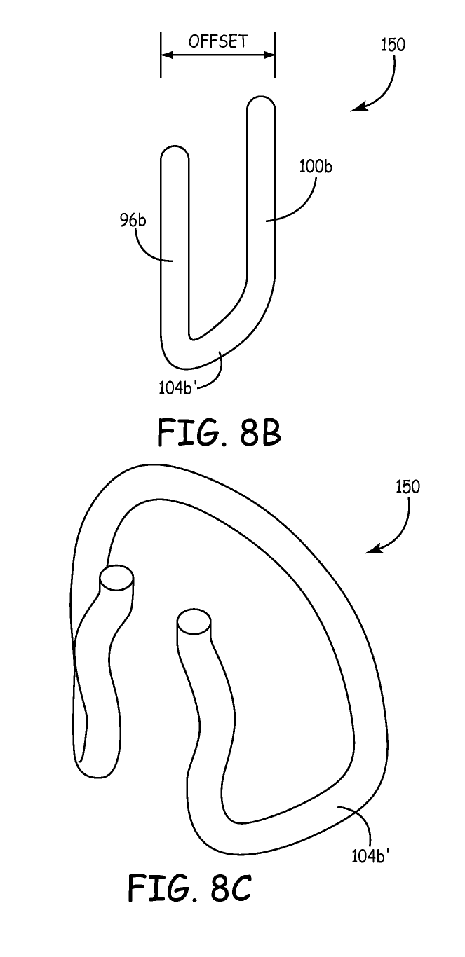

FIGS. 8A-8C are schematic diagrams illustrating another example spring clip 150. Spring clip 150 conforms substantially to spring clip 900 of FIGS. 5A-5C except that inner contour 72′ of spring clip 150 is non-coplanar with outer contour 74′ of spring clip 150. Instead, inner contour 72′ of spring clip 150 is offset in a direction along the longitudinal axis of electrode shaft 50. For example, inner contour 72′ may lie in a plane that is substantially parallel to the plane in which outer contour 74′ lies, but offset toward the distal end of the lead or toward the proximal end of the lead. In one example, the offset may be between 300-400 thousandths of an inch. However, the offset may be smaller or larger than 300-400 thousandths of an inch. Having the offset provides less torque for a fixed displacement.

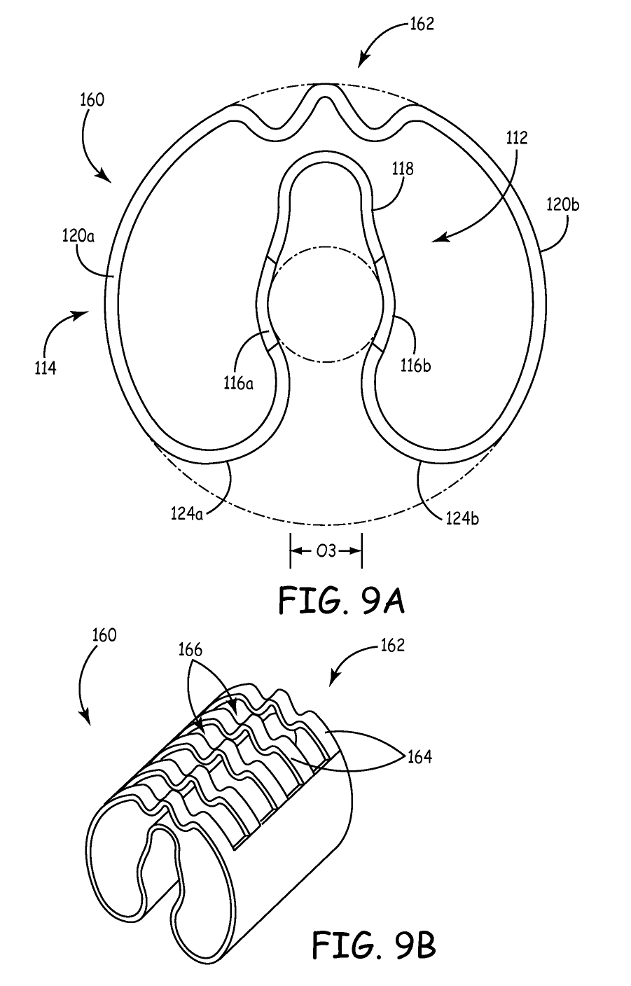

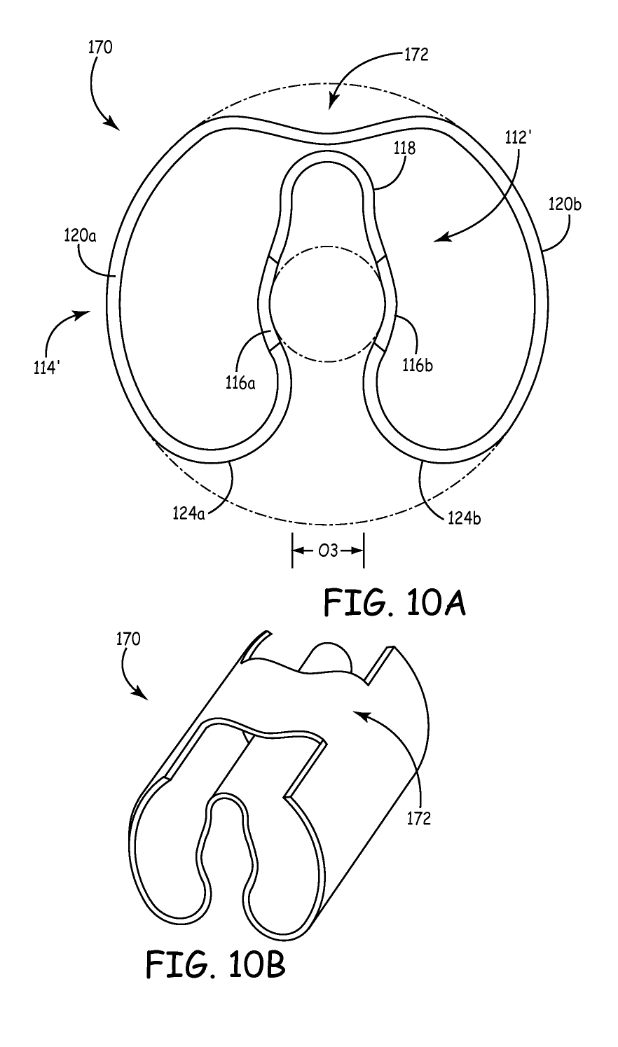

In the examples illustrated in FIGS. 4A-4C, FIGS. 5A-5C, FIGS. 6A-6C, FIG. 7 and FIGS. 8A-8C, the spring clips are designed such that a gap exists somewhere along the spring clip, e.g., in the inner contour of the spring clip, the outer contour of the spring clip or at a transition point from the inner contour to the outer contour. In other examples, however, there may not be a gap exists between open ends of segments anywhere along spring clip. Instead, a portion of the spring clip may be designed to provide some of the advantages of a gap, without actually having a gap between open ends of segments of the spring clip. Some examples of such spring clips are illustrated in FIGS. 9A and 9B and FIGS. 10A and 10B.

FIG. 9A and 9B illustrate another example spring clip 160 that may be used to provide contact between two structures. FIG. 9A is a front view of spring clip 160 and FIG. 9B is an angled view of spring clip 160. Spring clip 160 may correspond with spring clip 60 of FIG. 3. Spring clip 160 of FIGS. 9A and 9B may have generally the same shape as spring clip 110 of FIGS. 6A and 6B except that spring clip 160 does not have a gap between open ends of segments along the inner or outer contours. Instead of a gap between open ends of contact segments 120a and 120b, spring clip 160 includes a relief segment 162 that relieves at least a portion of the forces produced by insertion of electrode shaft 50 into inner contour 112 or placing energy dissipating structure 40 around the outer contour 114.

In the example illustrated in FIGS. 9A and 9B, relief segment 162 includes a plurality of sinusoidal segments 164 spaced apart from one another via spaces 166. Relief segment 162 may include more or few sinusoidal segments 164. In one example, relief segment 162 may include only one sinusoidal segment 164. Relief segment 162 may have a variable cross-sectional area relative to the rest of the coupling mechanism. For example, the total width of the sinusoidal segments 164 along the width is less than a width of the conductive portions of the rest of the coupling mechanism. In other words, the total length of conductive material across the width of relief segment 162 is less than the length of conductive material across the width of other portions of spring clip 160, e.g., due to spaces 166. Relief segment 162 may, in one example, have a width that is less than or equal to approximately half of the width of the rest of the spring clip 160. In another example, the width of relief segment 162 is less than or equal to approximately one-third of the width of the rest of the spring clip 160. Additionally the width of sinusoidal segments 164 may be smaller or larger than illustrated.

Relief segment 162 is illustrated as being sinusoidal for example purposes only. Relief segment 162 may be formed in a different shape, such as one or more triangle wave segments, arc segments, serpentine segments, accordion-like segments or any other geometry that is designed to be more susceptible to deformation that the other segments of spring clip 160. Typically, however, relief segment 162 has a geometry different than the rest of the spring clip 160.

When spring clip 160 is in the compressed state (e.g., included within an assembled lead), relief segment 162 may be more susceptible to elastic deformation than other portions of spring clip 160. As such, relief segment 162 may elastically deform to relieve at least some of the stress and/or strain of the other segments of spring clip 160 when in the compressed state. This stress and/or strain relief may also reduce the amount of force exerted by inner contour 112 on electrode shaft 50 such that the torque created by the forces is small enough to allow tip electrode 36 to be extended and retracted from the lead. In one example, forces F1 and F2 should create a torque that is less than or equal to approximately 0.15 inch-ounces. In another example, forces F1 and F2 should create a torque that is less than or equal to approximately 0.03 inch-ounces. In a further example, forces F1 and F2 should create a torque that is less than or equal to approximately 0.003 inch-ounces.

FIG. 10A and 10B illustrate another example spring clip 170 that may be used to provide contact between two structures. FIG. 10A is a front view of spring clip 170 and FIG. 10B is an angled view of spring clip 170. Spring clip 170 may correspond with spring clip 60 of FIG. 3. Spring clip 170 of FIGS. 10A and 10B may have generally the same shape as spring clip 110 of FIGS. 6A and 6B except that spring clip 170 does not have any gaps along the inner or outer contours. Instead of a gap between contact segments 120a and 120b, spring clip 170 includes a relief segment 172 that relieves at least a portion of the stress and/or strain produced by insertion of electrode shaft 50 into inner contour 112 or placing energy dissipating structure 40 around the outer contour 114.

In the example illustrated in FIGS. 10A and 10B, relief segment 172 is an arc segment having a width that is less than a width of other segments of spring clip 170. Relief segment 172 may, for instance, have a width that is less than or equal to approximately one-third (⅓) of the width of the other segments of spring clip 170. However, relief segment may have a width that is smaller by other factors, e.g., less than or equal to approximately one-half (½) of the width of the other segments of spring clip 170. As such, relief segment 172 may be more susceptible to elastic deformation than other portions of spring clip 170 when in the compressed state. Relief segment 172 may therefore elastically deform to relieve at least some of the stress and/or strain of the other segments of spring clip 170 when in the compressed state. This stress and/or strain relief may also reduce the amount of force exerted by inner contour 112 on electrode shaft 50 such that the torque created by the forces is small enough to allow tip electrode 36 to be extended and retracted from the lead. In one example, forces F1 and F2 should create a torque that is less than or equal to approximately 0.15 inch-ounces. In another example, forces F1 and F2 should create a torque that is less than or equal to approximately 0.03 inch-ounces. In a further example, forces F1 and F2 should create a torque that is less than or equal to approximately 0.003 inch-ounces.

Although relief segment 162 of FIGS. 9A and 9B and relief segment 172 of FIGS. 10A and 10B are illustrated as being located along the outside contours of spring clip 160 and 170, respectively, the relief segments may be located along inside contours of spring clips 160 and 170 or along a transition segment from outside contour 160 and 170. Moreover, spring clips in accordance with other embodiments may include more than one relief segment, such as one relief segment along the outer contour and one relief segment along an inner contour. Spring clips in accordance with other embodiments may both one or more relief segments as well as a gap.

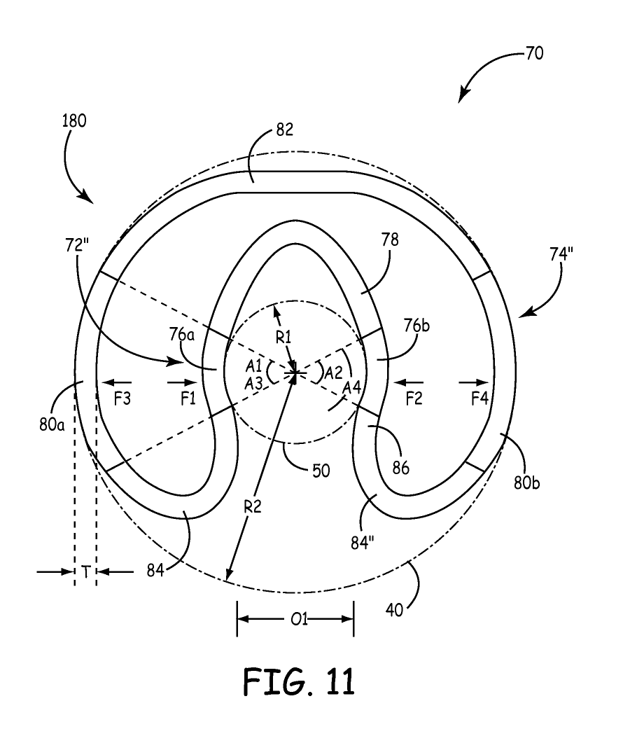

FIG. 11 is a schematic diagram illustrating a front view of another example spring clip 180. Spring clip 180 conforms substantially to spring clip 70 of FIGS. 4A-4C except that spring clip 130 does not have a gap or break anywhere along the inner contour or outer contour. Instead, spring clip 130 includes a segment 84″ that connects contact segment 76b of inner contour 72″ to contact segment 80b of outer contour 74″. Segment 84″ may be similar to segment 84 of spring clip 70 of FIGS. 4. Spring clip 180 also does not have a relief segment as described above with respect to FIGS. 9 and 10.

Although illustrated as conforming substantially with spring clip 70 of FIGS. 4, spring clip 180 may alternatively conform with the designs illustrated in FIGS. 5-7 without a gap or break along the inner and outer contours. Additionally, spring clips may be formed with no gap or break along the inner and outer contours and with offsets similar to that illustrated in FIG. 8. In other words, spring clip 180 of FIG. 11 may have inner and outer contours that are non-coplanar.

Additionally, spring clips of other shapes may be formed with no gaps, but with an opening that allows the electrode shaft to be inserted within the inner contour from the side instead of being slid on from one of the ends of the electrode shaft. This provides lead construction advantages to coupling mechanism that must be slid onto electrode shaft from one of the ends of the electrode shaft, e.g., as is the case for coupling mechanism having outer contours that extend around an entire circumference with no opening along the side.

It is understood that the present disclosure is not limited for use in pacemakers, cardioverters or defibrillators. Other uses of the leads described herein may include uses in patient monitoring devices, or devices that integrate monitoring and stimulation features. In those cases, the leads may include sensors disposed on distal ends of the respective lead for sensing patient conditions. The leads described herein may be used with a neurological device such as a deep-brain stimulation device or a spinal cord stimulation device. In other applications, the leads described herein may provide muscular stimulation therapy, gastric system stimulation, nerve stimulation, lower colon stimulation, drug or beneficial agent dispensing, recording or monitoring, gene therapy, or the like. In short, the leads described herein may find useful applications in a wide variety medical devices that implement leads and circuitry coupled to the leads.

Various examples have been described. These and other embodiments are within the scope of the following claims. Additionally, skilled artisans appreciate that other dimensions may be used for the mechanical and electrical elements described herein. It is also expected that the teachings herein, while described relative to a bipolar lead, can also be applied to a unipolar lead or other multipolar configurations as well as co-radial and multi-lumen configurations. These and other examples are within the scope of the following claims.

1. A medical electrical lead comprising:
a lead body having a proximal end configured to couple to an implantable medical device and a distal end;
a conductive electrode shaft located near the distal end of the lead body;
a conductor that extends from the proximal end of the lead body and couples to the conductive electrode shaft,
an electrode located near the distal end of the lead body and electrically coupled to an opposite end of the conductive electrode shaft as the conductor;
an energy dissipating structure located adjacent the electrode, the energy dissipating structure including a conductive element; and
a coupling mechanism formed of a conductive material that contacts the conductive electrode shaft and the energy dissipating structure, wherein the conductive material is shaped to form:
an inner contour of the conductive material that receives the conductive electrode shaft and exerts a inward force on the conductive electrode shaft;
an outer contour of the conductive material that exerts an outward force on the energy dissipating structure; and
a relief segment somewhere along the coupling mechanism that is designed to be more susceptible to deformation due to forces than the rest of the coupling mechanism.

2. The medical electrical lead of claim 1, wherein the relief segment reduces forces on the conductive electrode shaft such that the conductive electrode shaft may be moved to extend the electrode.

3. The medical electrical lead of claim 1, wherein the relief segment has a variable cross-sectional area relative to the rest of the coupling mechanism.

4. The medical electrical lead of claim 1, wherein the relief segment comprises one of a sinusoidal segment, serpentine segment and an accordion-like segment.

5. The medical electrical lead of claim 1, wherein the relief segment has a width having a plurality of conductive portions along the width separated from one another by spaces.

6. The medical electrical lead of claim 1, wherein a width of conductive portions of the relief segment is smaller than a width of the conductive portions of the rest of the coupling mechanism.

7. The medical electrical lead of claim 6, wherein the width of the relief segment is less than or equal to approximately half of the width of the rest of the coupling mechanism.

8. The medical electrical lead of claim 6, wherein the width of the relief segment is less than or equal to approximately one-third of the width of the rest of the coupling mechanism.

9. The medical electrical lead of claim 6, wherein the relief segment has a geometry different than the rest of the coupling mechanism.

10. The medical electrical lead of claim 1, wherein a large portion of current induced on the conductor by high frequency signals is conducted to the energy dissipating structure via the coupling mechanism while a small portion of the current produced on the conductor by low frequency therapy signals is conducted to the energy dissipating structure.

11. The medical electrical lead of claim 10, wherein the at least approximately 80% of the current induced on the conductor by signals having a frequency greater than approximately 1 megahertz (MHz) is conducted to the energy dissipating structure.

12. The medical electrical lead of claim 11, wherein the less than approximately 5% of the current induced on the conductor by signals having a frequency of less than approximately 100 kilohertz (kHz) is conducted to the energy dissipating structure.

13. The medical electrical lead of claim 1, further comprising a layer of insulating material covering at least a portion of the surface of conductive element of the energy dissipating surface.

14. The medical electrical lead of claim 1, wherein the inner contour and outer contour of the coupling mechanism are coplanar.

15. The medical electrical lead of claim 1, wherein the relief segment is located along one of the outer contour of the coupling mechanism, the inner counter of the coupling mechanism, and a transition from the inner contour to the outer contour of the coupling mechanism.

16. The medical electrical lead of claim 1, wherein torque exerted on the conductive electrode shaft by the coupling mechanism is less than approximately 0.15 inch-ounces.

17. The medical electrical lead of claim 1, wherein the coupling mechanism has a thickness that is less than or equal to approximately 5 mil.

18. The medical electrical lead of claim 1, wherein the conductive material of the coupling mechanism has one of a substantially round geometry and a substantially flat geometry.

19. The medical electrical lead of claim 1, wherein the conductive material of the coupling mechanism includes at least one of platinum, platinum iridium, nickel-cobalt based alloy, titanium, tantalum, platinum clad tantalum, stainless steel, silver cored nickel-cobalt based alloy, silver cored tantalum, and niobium.

20. The medical electrical lead of claim 1, wherein the coupling mechanism includes a layer of capacitive or inductive material that covers at least a portion of the conductive material of the coupling mechanism.
